(19) United States
(12) Plant Patent Application Publication
(10) Pub. No.: US 2004/0148671 P1
(43) Pub. Date: Jul. 29, 2004

(54) BLUEBERRY PLANT DENOMINATED 'AURORA'

(76) Inventor: James F. Hancock, East Lansing, MI (US)

Correspondence Address: HARNESS, DICKEY & PIERCE, P.L.C., P.O. BOX 828, BLOOMFIELD HILLS, MI, 48303, US

(21) Appl. No.: 10/350,345

(22) Filed: Jan. 23, 2003

(51) Int. Cl. A01H 5/00

(52) U.S. Cl. PLT/157

(57) ABSTRACT

Aurora is a new blueberry variety of Vaccinium corymbosum. It is a productive, very late ripening variety with high fresh market quality intended for areas where northern highbush varieties are grown successfully. Plants of Aurora are vigorous and upright. Canes are numerous, moderately branched and the fruit are well exposed. Its berries are moderately large, have small, dry picking scars, excellent powder-blue color, delicious flavor and excellent firmness. Aurora has a harvest season that begins about 5 days after the variety known as “Elliott.”

### CROSS-REFERENCE TO RELATED APPLICATIONS

This application is related to the present inventor's United States patent applications entited “Blueberry plant denominated ‘Liberty,’” filed on even date as Attorney Docket No. 6550-000074, and “Blueberry plant denominated ‘Draper,’” filed on even date as Attorney Docket No. 6550-000075. Both of these applications are also assigned to the assignee of the present application. The disclosures of the above applications are hereby incorporated herein by reference.

### LATIN NAME AND VARIETY DENOMINATION

The present invention relates to a new and distinct variety of Vaccinium corymbosum, which is hereby denominated “Aurora.”

### SUMMARY OF THE INVENTION

The present invention relates to a new and distinct variety of highbush blueberry plant, denominated “Aurora.” Aurora is a new blueberry variety of Vaccinium corymbosum from the Michigan State University breeding program. It is a productive, very late ripening variety with high fresh market quality that is intended for areas where northern highbush varieties are grown successfully. Plants of Aurora are vigorous and upright. Canes are numerous, moderately branched and the fruit are well exposed. Its berries are moderately large, have small, dry picking scars, excellent powder-blue color, delicious flavor and excellent firmness. Aurora has a harvest season that begins about 5 days after the variety known as “Elliott.” Blueberry growers often receive their highest profits in the late season. Aurora will provide a new late ripening option with high quality fruit.

Emasculated flowers of the “Brigetta” highbush blueberry from Australia were pollinated in 1991 with pollen from the northern highbush variety Elliott. The seeds were germinated, grown in a greenhouse for 1 year and then field planted at Benton Harbor, Mich. Aurora was selected from a group of 54 siblings in 1997. The original selection has been evaluated at Benton Harbor, Mich. annually for 11 years. Aurora has been propagated by hardwood cuttings that produced over a hundred shoots that were rooted in the greenhouse and then planted in the field. In addition, Aurora has been propagated by softwood cuttings that have been rooted. Furthermore, generation of micro-shoots in a greenhouse using established tissue culture methods has produced thousands of clones of Aurora. Such methods are discussed in the following references, which are incorporated by reference in their entirety: Doran, W. L. and Bailey, J. S. “Propagation of the high bush blueberry by softwood cuttings,” Bulletin Massachusetts Agricultural Experiment Station; no. 410. Amherst, Mass. Massachusetts State College, 1943; Doehlert, C. A. “Propagating blueberries from hardwood cuttings,” Circular (New Jersey Agricultural Experiment Station) 490. New Brunswick, N.J. New Jersey Agricultural Experiment Station, 1945; Doehlert, C. A. “Propagating blueberries from hardwood cuttings,” Circular (New Jersey Agricultural Experiment Station) 551. New Brunswick, N.J.: New Jersey Agricultural Experiment Station, 1953; El Shiekh, A.; Wildung, D. K.; Luby, J. J.; Sargent, K. L.; Read, P. E. “Long term effects of propagation by tissue culture or softwood single node cuttings on growth habit, yield, and berry weight of ‘Northblue’ blueberry,” Journal of the American Society for Horticultural Science. 1996, 121: 2, 339 342; Galletta, G. J.; Ballington, J. R.; Daubeny, H. A.; Brennan, R. M.; Reisch, B. J.; Pratt, C.; Ferguson, A. R.; Seal, A. G.; McNeilage, M. A.; Fraser, L. G.; Harvey, C. F.; Beatson, R. A.; Hancock, J. F.; Scott, D. H.; Lawrence, F. J.; Janick, J. (ed.); Moore, J. N. “Fruit breeding. Volume II. Vine and small fruits,” Department of Horticulture, Purdue University, West Lafayette, Ind. 1996 John Wiley and Sons; New York; USA; Strik, B.; Brun, C.; Ahmedullah, M.; Antonelli, A.; Askham, L.; Barney, D.; Bristow, P.; Fisher, G.; Hart, J.; Havens, D. Draper A. D. and Chandler C. K. “Accelerating highbush blueberry selection evaluation by early propagation,” Journal of the American Society for Horticultural Science. 1986 111(2): 301-303; Pritts M. P. and Hancock J. F. (Eds.) “Highbush blueberry production guide,” Northeast Regional Agricultural Engineering Service, Ithaca, N.Y., USA 1992.

Clones of Aurora have been tested for two years at Grand Junction, Mich., South Haven, Mich., Lacota, Mich., Corvallis, Oreg., and Lowell, Oreg. In all cases all resulting plants have stably displayed characteristics of the variety. Consistent high yields at Benton Harbor and Grand Junction, Mich. indicate that the buds and wood of Aurora are tolerant to fluctuating late fall and spring temperatures. Aurora also has excellent winter hardiness, as it has routinely been challenged with mid-winter temperatures below −20 C. Probable areas of adaptation and markets include blueberry growers in Michigan and across the USA, Canada, Argentina, Australia, Chile, France, Germany, and New Zealand.

### BRIEF DESCRIPTION OF THE DRAWINGS

The present invention will become more fully understood from the detailed description and the accompanying drawings, wherein:

FIG. 1 is a photographic print in full color showing, in the foreground, an exemplary 4-year-old Aurora blueberry plant. The plants appearing in the background are of a different variety and are not part of the Aurora blueberry plant, nor is the grass appearing in the picture part of the Aurora blueberry plant.

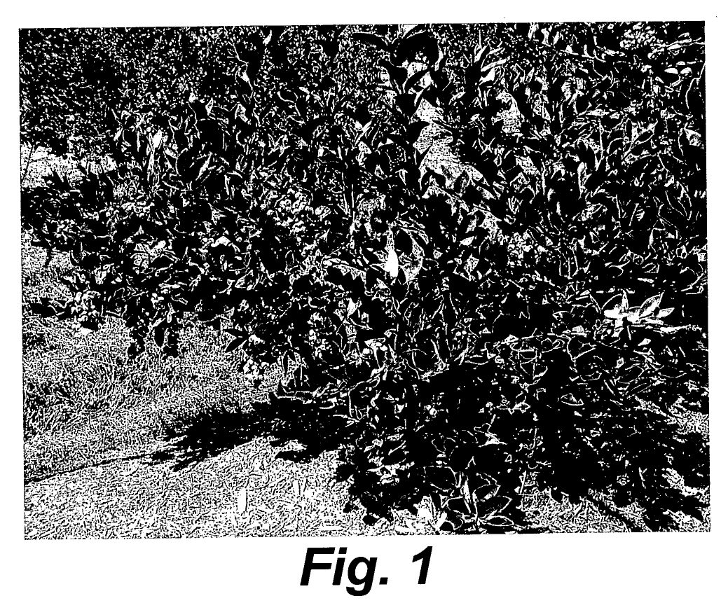

FIG. 2 is a photographic print in full color illustrating exemplary fruit clusters of a 4-year-old Aurora blueberry plant. Some of the fruit is not yet mature and does not show the characteristic mature color.

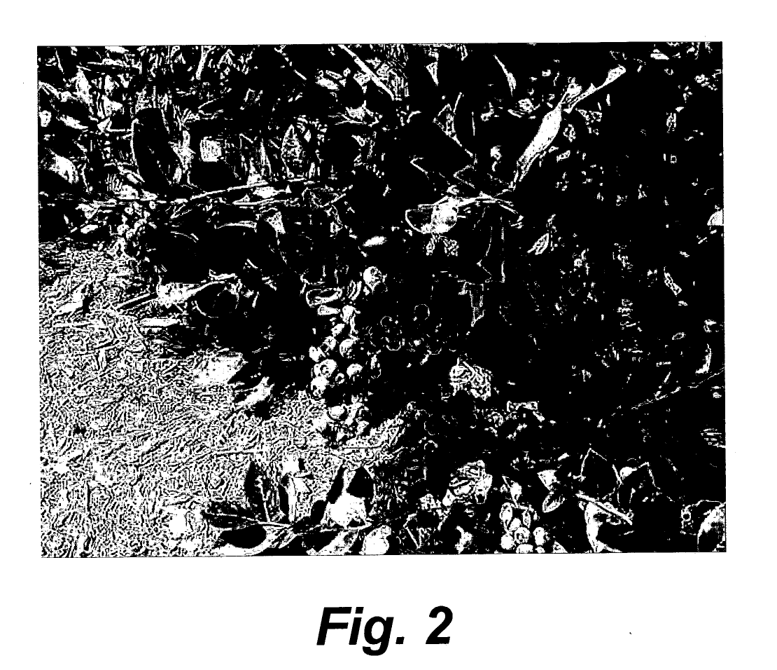

### DETAILED BOTANICAL DESCRIPTION

The following is a detailed botanical description of the new and distinct variety of blueberry, its flowers, fruit and foliage, based on observations of specimens grown at Benton Harbor Mich., Grand Junction, Mich., South Haven, Mich., Lacota, Mich., Corvallis, Oreg., and Lowell, Oreg., over a two year period. The taxonomic characteristics chosen in the detailed description are standard in the practice (R E Gough, R J Hindle, and V G Shutak, “Identification of Ten Highbush Blueberry Cultivars using Morphological Characteristics,” HortScience 11 (5):512-4, 1976). Color descriptions, except those given in common terms, are presented in Royal Horticultural Society Colour Chart designations. In cases where the color descriptions cited from The Royal Horticultural Society Colour Chart differ from the colors shown in the drawings, the colors cited from The Royal Horticultural Society Colour Chart should be considered accurate. Any deviation from these colors in the drawings is due to failure of the photographic process to exactly duplicate the colors of nature. In addition, fruit color designations in Table I are applicable only to mature fruit.

TABLE I
Aurora Characteristics

| Characteristic | Aurora |
|---|---|
| Bush | |
| Mature height | 1.5 m |
| Diameter/width | 1.1 |
| Growth habit | Upright to semi-spreading |
| Annual renewal canes | 5-6 |
| Internode length on | 2.5 cm |
| spring shoots | |
| Mature cane color | Greyed-Green (198A) |
| Fall color-new shoots | Patches of Red-Purple |
| | (60A) |
| Foliage | |
| Leaf shape | Elliptic-ovate |
| Leaf length/width | 2.1 |
| Leaf serration | Entire |
| Pubescence | None |
| Color-top | Green (137A) |
| Color-bottom | Green (138A) |
| Petiole length | 4 mm |
| Blossoms | |
| Shape of corolla | Elongate-urceolata |
| Calyx | 5 lobed |
| Style length | At edge of corolla |
| Color of open flower | Mostly white |
| Flower # per cluster | 8-10 |
| Mature Fruit | |
| Shape | Globose, uniform |
| Color with bloom | Violet Blue (98A) |
| Color without bloom | Blue (103A) |
| Pedicel scar size | 1.44 mm |
| Average weight | 1.5 gm |

In trials in Michigan and Oregon, Aurora has consistently had better color, was firmer and had a better picking scar than Elliott (Tables II and III). They also had greatly improved flavor. In several post-harvest trials, the relative fruit rot susceptibility of the Elliott and Aurora genotypes was similar and good (Table IV). Aurora had a slightly longer storage life than ‘Elliott’ in 2001 and 2002.

TABLE II
Characteristics of mature ‘Aurora’ in comparison
to ‘Elliott’ at Benton Harbor, MI in 2001 and 2002.
All values were the same in both years, except for
harvest date. The rating scale was 1-9, with
1-4 = inferior, 5-6 = acceptable,
7 = good, 8 = very good and 9 = superior.

| Traits | Aurora | Elliott |
|---|---|---|
| Horticultural | | |
| Date of first harvest | 8/29 and 9/7 | 8/23 and 8/27 |
| Fruit load | 7 | 8 |
| Size | 7 | 6 |
| Color | 8 | 6 |
| Picking scar | 8 | 7 |
| Firmness | 8 | 7 |
| Flavor | 8 | 5 |
| Taxonomy | | |
| Leaf margin | entire | entire |
| Leaf shape | elliptic-ovate | elliptic |
| Leaf length/width ratio | >2 | <2 |
| Bush diameter/width | 1.1 | 1.4 |
| ratio | | |
| Fall color on 1-year | red and | solid red |
| shoots | green | |
| Bark texture-mature | rough | rough |
| wood | | |

TABLE III
Mean fruit ratings of ‘Elliott’ and ‘Aurora’ at Grand Junction, MI,
Corvallis, OR and Lowell, OR in 2002. Plants were set as
two-year-old rooted cuttings in 2000. Evaluations were
made when the bushes were 50% ripe.

| | | Fruit | | | Picking | | |
|---|---|---|---|---|---|---|---|
| Location | Cultivar | load | Size | Color | scar | Firmness | Flavor |
| Grand | Elliott | 8^{z} | 6 | 6 | 7 | 7 | 5 |
| Junction | Aurora | 7 | 7 | 8 | 8 | 8 | 8 |
| Corvallis | Elliott | 9 | 6 | 7 | 7 | 7 | 6 |
| | Aurora | 8 | 7 | 8 | 8 | 8 | 8 |
| Lowell | Elliott | 9 | 6 | 7 | 7 | 7 | 6 |
| | Aurora | 8 | 7 | 8 | 8 | 8 | 8 |

^{z}The rating scale 1-9, with 1-4 = inferior, 5-6 = acceptable, 7 = good, 8 = very good and 9 = superior.

TABLE IV
Comparison of postharvest storage life and fungal rots of ‘Elliott’ and
‘Aurora’ harvested at Benton Harbor, MI. Fruit were picked on
the same day, when the bushes were 30-40% ripe.

| Determinations | Elliott | Aurora |
|---|---|---|
| Percentage of fruit rotted^{X} | | |
| 2000 | 12% | — |
| 2001 | 12% | 11% |
| 2002 | 10% | 16% |
| Types of fruit rots in 2002^{Y} | | |
| Alternaria | 6 | 10 |
| Colletotrichum | 4 | 2 |
| Botrytis | 0 | 0 |
| Storage life (days)^{Z} | | |
| 2000 | 7 | — |
| 2001 | 7 | 14 |
| 2002 | 7 | 14 |

^{X}Four pints of fruit were evaluated after being held for 3 weeks at 2 C. in zip-lock storage bags. Fungal species were not determined.
^{Y}Fifty fruit were randomly selected from 4 pints and held for ten days at room temperature.
^{Z}Four pints of fruit were held at 2 C. in plastic zip-lock bags and examined at 7 day intervals. The containers were considered non-salable, if more than a few fruit appeared rotten or > 25% were soft to the touch.

What is claimed is:

1. A new and distinct highbush blueberry plant, substantially as illustrated and described herein.
